(19) United States
(12) Patent Application Publication
(10) Pub. No.: US 2024/0382530 A1
(43) Pub. Date: Nov. 21, 2024

(54) DEVICE FOR PREPARING LIPOSUCTION FAT FOR AUTOLOGOUS FAT TRANSFER

(71) Applicant: LABORATOIRE BENEWMEDICAL, MELESSE (FR)

(72) Inventors: Noël Laurent, BONNEMAIN (FR); Julien Terrier, MONACO (MC); Xavier Migaud, RENNES (FR)

(21) Appl. No.: 18/689,989

(22) PCT Filed: Sep. 6, 2022
(86) PCT No.: PCT/EP2022/074702

§ 371 Date: Mar. 7, 2024

(30) Foreign Application Priority Data
Sep. 8, 2021 (FR) 2109393

(51) Int. Cl. A61K 35/35; A61M 1/00

(52) U.S. Cl. CPC A61K 35/35; A61M 1/892; A61M 1/895; A61M 2202/08

(57) ABSTRACT

A device for preparing fat harvested from a first area of a patient's body for reinjection into a second area of the patient's body. The device includes: a structure having first and second branches to which first and second syringes are connected; and a connecting element which fits into the structure, connects the two branches, and has a membrane for filtering the fat. The connecting element is connected to a third syringe and assumes at least: an emulsification position forming with the first and second branches a first circulation circuit for the fat between the first syringe and the second syringe; and a transfer position forming with the first or second branches a second circulation circuit for the fat between the first or second syringe and the third syringe.

### 1. FIELD OF THE INVENTION

The field of the invention is that of plastic surgery, more particularly cosmetic or reconstructive surgery.

More specifically, the invention relates to the general principle of “lipofilling”, or lipostructure, cosmetic or reconstructive surgery procedures consisting of injecting fat taken from an area of the patient's body where it is in excess in order to reinject it into another area of the patient's body where it is lacking.

In particular, the invention relates to a device for emulsifying and filtering fat taken from a patient for autologous transfer.

### 2. PRIOR ART AND ITS DISADVANTAGES

Lipostructure is a technique in cosmetic or reconstructive surgery which involves taking small quantities of fat from a patient's body and reinjecting it into the patient for the purpose of filling or remodelling. The lipostructure procedure is also known as autologous fat transfer or the autografting of fatty tissue.

The lipostructure technique involves two main steps performed in series. In the first step, referred to as fat harvesting, fat is removed and recovered from one or more fat sites on a patient's body, such as the thighs or stomach, by a number of known techniques such as liposuction or lipoplasty.

The harvested fat is a complex mixture comprising of several components. In particular, the harvested fat is composed of mature adipocytes, precursor adipocytes, other precursor cells and lipids from ruptured mature adipocytes. Adipocytes are the primary cells of adipose tissue, which is the loose connective tissue of the body where most of the fat is located. As connective tissue, the adipose tissue also includes fibres, fragments of fibres and other non-fatty materials in addition to adipocytes. Lipids are molecules, including fat molecules which can be characterised in simple terms as free fat or fat particles.

The harvested fat can be classified by size as macro-fat, micro-fat or nano-fat, in descending order of particle size.

In the second step, known as fat reinjection, the harvested fat is reinjected into the body of the same patient in small quantities into one or more target areas at two or three depth levels (periosteum, muscles and hypodermis).

The reinjected fat increases the volume of the treated reinjection site and improves the patient's appearance. Autologous fat grafting has clear advantages: no allergic reactions, correction of large volumes and improvement of the trophicity of the surrounding skin.

Nano-fat, compared to larger fats, has proved to be more suitable for fat reinjection as it produces significantly better results in terms of the patient's final appearance. Nano-fat corresponds to emulsified and filtered fatty tissue with a high concentration of stem cells. To obtain a purer nano-fat with a high concentration of stem cells from the harvested fat, the harvested fat needs to be broken down and sized before being reinjected into the patient's body.

There are currently several different techniques for emulsifying and filtering liposuctioned fat to obtain a nano-fat composed mainly of Adipose Derived Stem Cells (ADSC).

These techniques are generally based on emulsification then filtration using a system comprising either a single emulsification/filtration device, or several different devices, used in succession to emulsify then filter the fat.

Generally, the emulsification step is performed by successive back and forth movements (for example 30 back and forth movements) of the harvested fat between two syringes connected to one another by a fat preparation device. More specifically, once the fat has been harvested by liposuction, it is prefiltered to obtain micro-fat. This is then placed into a first syringe which is then connected to a second syringe by a fat preparation device. The practitioner then applies force to a plunger of the first syringe so that the fat passes through the fat preparation device into the second syringe. This action is then repeated to make the fat pass back from the second syringe to the first syringe, and so on, thus achieving the desired emulsification.

The emulsified fat can be filtered by successive filtrations through different devices comprising filtering elements of different sizes to obtain different sizes of fat. Successive passages of the fat through these filtering elements of different sizes makes it possible to obtain smaller and smaller fat sizes until nano-fat is obtained.

One disadvantage of using different devices in succession to obtain nano-fat, is that it is necessary to pass the fat from one device to the other, which increases the risk of contaminating the fat.

A device is also known for preparing the fat harvested by liposuction, configured to perform the emulsification and filtration on its own. This device, marketed under the name HY-TISSUE NANOFAT®, consists of a reservoir, or flexible plastic bag, comprising a filter, and a T-shaped element. More specifically, the two syringes used to emulsify the harvested fat are connected to the device at two branches of the T-shaped element. In particular, they can be positioned either at 180° to one another (at the two opposite upper branches of the T), but also at 90° (one at an upper branch of the T and the other at the lower branch or foot of the T).

The reservoir is then connected to the device at the third branch of the T-shaped element.

One disadvantage of the HY-TISSUE NANOFAT® device is that its reservoir is not completely rigid by nature (flexible plastic bag). Thus, the action of gravity causes it to assume a vertical position, and therefore not to remain in the linear extension of the branch of the T-shaped element to which it is connected, and even to “be knocked about” by the actions applied by the practitioner to the T-shaped element to perform the emulsification. This break in the angle and this induced movement relative to the T-shaped element cause a risk of detachment of the reservoir from the T-shaped element, as well as obstruction and discomfort during the repeated gestures by the practitioner for the emulsification.

Furthermore, the 180° angle between the two syringes for the emulsification of the fat means that the practitioner's hands and wrists are not positioned ergonomically during the emulsification step, which makes the operation uncomfortable and quickly tiring for the practitioner.

There is therefore a need to provide an emulsification and filtration technique for fat harvested by liposuction before being reinjected which is simpler and more ergonomic for the practitioner.

### 3. PRESENTATION OF THE INVENTION

The invention addresses this need by proposing a device for the preparation of fat harvested from a first area of a patient's body for reinjection into a second area of the patient's body. This preparation device comprises:
a structure comprising a first and a second branch to which a first syringe and a second syringe are connected respectively,
a connecting element of the first and second branches, this connecting element fitting into the structure.

The connecting element is connected to a third syringe, and is configured to assume at least two positions:
an emulsification position in which the connecting element forms with the first and second branches of the structure a first circulation circuit for the fat between the first syringe and the second syringe,
a transfer position in which the connecting element forms with the first or second branches of the structure a second circulation circuit for the fat between the first or the second syringe and a third syringe, and the connecting element comprises a membrane for filtering the fat.

Thus, the invention proposes a completely novel and inventive approach for preparing fat harvested from a patient before it is reinjected. It is based on a single “all-in-one” device whose original configuration makes it possible to successively emulsify then filter the harvested fat. The preparation device according to the invention makes it possible to directly connect up to three syringes: two syringes for the emulsification operation, and a filtration and recovery cell. In other words, the fat harvested in a first syringe can be directly emulsified by passing it successively between the first and second syringe, then filtered by passing through a filtering membrane towards the third syringe.

The possibility of emulsifying, then filtering without having to change device is provided by the connecting element (hereinafter the connecting element is also referred to as a pin), which, according to the invention, not switches only from the first emulsification circuit to the second filtering and transfer circuit, but also integrates the filtering function of the emulsified fat and connects the third fat recovery syringe. When the connecting element is in the emulsification position, the first emulsification circuit allows the fat to pass between the first and the second syringes through the branches of the structure supporting these syringes. When the connecting element is in the transfer position, the second circuit connects the first or second syringe to the third one through the filtering membrane and ends in the third recovery syringe.

Integrating the filtering membrane directly into the connecting element has the advantage of making the device more compact.

The rigid and “all-in-one” nature of the device according to the invention makes it easier to handle. Furthermore, the risks of leaks are reduced, as are the risks of contamination of the fat, as the fat is handled in a closed circuit.

According to one feature of the invention, the first and second branches of the structure are positioned in a V shape relative to one another.

One advantage of this V shape of the two branches of the structure is that it improves the ergonomics of the device and simplifies its use by the practitioner. It simplifies the actions of the practitioner during the emulsification phase by making the positioning of the practitioner's hands and wrists more ergonomic.

According to another feature of the invention, the first and second branches of the structure are positioned relative to one another at an angle (α) of between 45° and 135°, preferably 90°.

The practitioner's hands and wrists are therefore positioned relative to one another in a more ergonomic manner which simplifies their movements and therefore reduces the risk of injury.

According to another feature of the invention, the first and second branches of the structure form a Y shape with the connecting element.

An advantage of this Y shape is that it provides a more ergonomic and natural grip. It facilitates the emulsification of fat between the two upper branches of the structure while allowing easy recovery of the filtered fat from the bottom of the device, i.e. from by the connecting element forming a third lower branch of the preparation device.

Furthermore, the fact that the branches of the structure used for the emulsification are associated with a third branch formed by the connecting element which incorporates a filtering membrane makes it possible to reinforce the solidity of the device but also to simplify its use and operation.

According to a particular aspect of the invention, the first branch forms a first angle (β_{A}) with the connecting element of approximately 112.5°-157.5°, preferably 135°, and the second branch forms a second angle (β_{B}) with the connecting element of approximately 112.5°-157.5°, preferably 135°.

According to another particular aspect of the invention, the filtering membrane is held in the connecting element by means of a cover fixed to the connecting element. This cover comprises a port for connecting with the third syringe.

Advantageously, the cover is fixed to the connecting element, thus making it possible to create a closed chamber therewith in which the filtering membrane is installed. Furthermore, one side of the cover has a Luer-type threaded end piece onto which the third syringe can be screwed. It is thus possible to use a single device to emulsify the harvested fat and then filter it. This all-in-one handling makes it easier to prepare the fat prior to reinjection into a patient and to limit the risk of it becoming contaminated.

According to one feature of the invention, the structure comprises at least one track comprising a first end for engaging the emulsification position and a second end for engaging the transfer position and the connecting element comprises a base with at least one fixing stud, said at least one fixing stud being adapted to fit and slide in said at least one track until it locks into either of the first and second ends, when the connecting element is placed in the corresponding position.

Advantageously, the fixing studs make it possible both to move the connecting element relative to the structure and to hold this connecting element reversibly in either of the positions. Advantageously, the fixing studs make it possible to prevent the connecting element from inadvertently changing position during the emulsification step or during the transfer step, thereby preventing these steps from being carried out correctly.

According to another feature of the invention, the connecting element comprises a central stud fixed to the base and oriented in a longitudinal (y) axis of the structure, the central stud comprising at least two movement transformation interfaces into which at least one follower of the first branch and at least one follower of the second branch of the structure slide to transform a rotational movement of the connecting element relative to the structure, by the effect of a rotational action of the connecting element exerted by a practitioner on the base about the longitudinal axis (y), into a translational movement of the connecting element along the longitudinal axis (y). In an advantageous manner, the passage from an emulsification position to a transfer position is achieved very simply by the practitioner rotating the connecting element about a vertical longitudinal axis. The rotational movement of the connecting element is transformed simultaneously into a translational movement along this longitudinal axis (or vertical axis “y” in a standard orthonormal frame of reference) by way of the cooperation between movement transformation interfaces located on its central stud and followers located on the branches of the structure. Depending on the direction of rotation selected, this displacement of the connecting element makes it possible to form the first or second fat circulation circuit.

According to another feature of the invention, the central stud further comprises:
a first fat emulsification channel intended to form with the first and second branches of the structure the first circulation circuit of fat between the first syringe and the second syringe, when the connecting element is placed in the emulsification position,
a second fat transfer channel intended to form with the first or second branches of the structure the second circulation circuit of fat between the first or second syringe and third syringe, when the connecting element is placed in the transfer position.

The invention also relates to a method for preparing fat harvested from a first area of a patient's body for reinjection into a second area of the patient's body. This method uses the preparation device as described above and comprises the following steps:
positioning the connecting element in the emulsification position;
emulsifying the harvested fat by successive and repeated pressing movements by an operator on the first and second syringes, the pressing movements causing the circulation of fat in the first circuit from the first syringe to the second syringe and vice-versa;
positioning the connecting element in the transfer position by changing the position of the connecting element relative to the structure;
filtering and transferring the fat emulsified by operator pressing on one of the first or second syringes, the pressing movement causing the harvested fat to flow in the second circuit through the filtering membrane into the third syringe.

### 4. BRIEF DESCRIPTION OF THE FIGURES

Other aims, features and advantages of the invention will become clearer from the following description, given solely by way of an illustrative example, without being limiting, in relation to the Figures, in which:

FIG. 1 shows a diagram of an exploded view of the various elements forming the fat preparation device according to one embodiment of the invention;

FIG. 2 shows a diagram of the body of fat preparation device according to one embodiment of the invention;

FIG. 3A shows a diagram of the pin of fat preparation device according to one embodiment of the invention;

FIG. 3B shows a diagram of a longitudinal cross-section of the pin of the preparation device according to one embodiment of the invention;

FIG. 4 shows a diagram of a cover of the fat preparation device 15 according to one embodiment of the invention;

FIG. 5 shows a diagram of the fat preparation device in an emulsification position according to one embodiment according to one embodiment of the invention;

FIG. 6 shows a diagram of the fat preparation device in transfer position according to one embodiment of the invention;

FIGS. 7A to 7E show the steps of the emulsification method using the fat preparation device according to one embodiment of the invention.

### 5. DETAILED DESCRIPTION OF THE INVENTION

The invention relates to a device for the preparation of fat harvested by liposuction to obtain nano-fat for an autologous transfer of the latter to a patient. More particularly, the invention relates to a fat preparation device configured to emulsify and filter fat which is more ergonomic and simpler to use than the devices known from the prior art. Furthermore, the fat preparation device according to the invention makes it possible to limit any external contamination of the fat by proposing emulsifying and filtering the fat in a closed circuit, i.e. without changing the preparation device during the procedure.

To achieve this, the fat preparation device according to the invention connects a plurality of standard syringes together and can shift reversibly from a first emulsification position to a second transfer position of the emulsified fat. It is thus possible to recover better quality nano-fat in a very simple way and with a minimum number of operations for the practitioner.

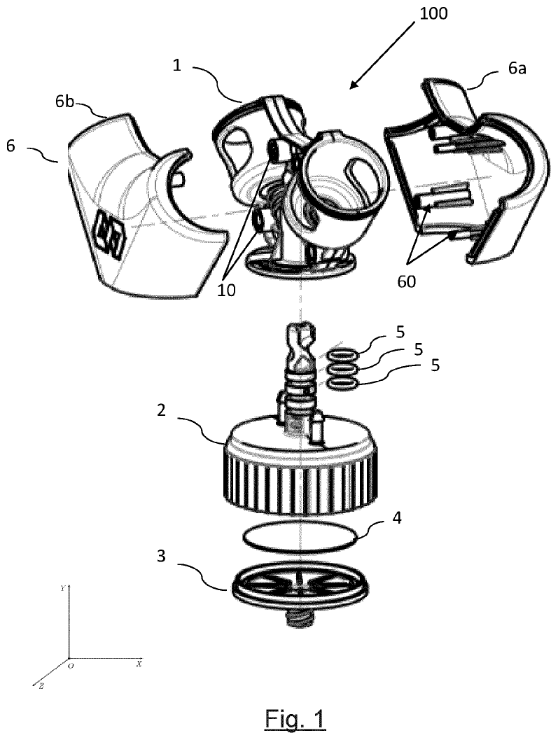

An exploded view of the various elements forming the fat preparation device according to one embodiment of the invention is now explained with reference to FIG. 1.

The fat preparation device 100 comprises a plurality of separate elements which fit together reversibly or irreversibly. Once assembled, these different elements form the preparation device 100.

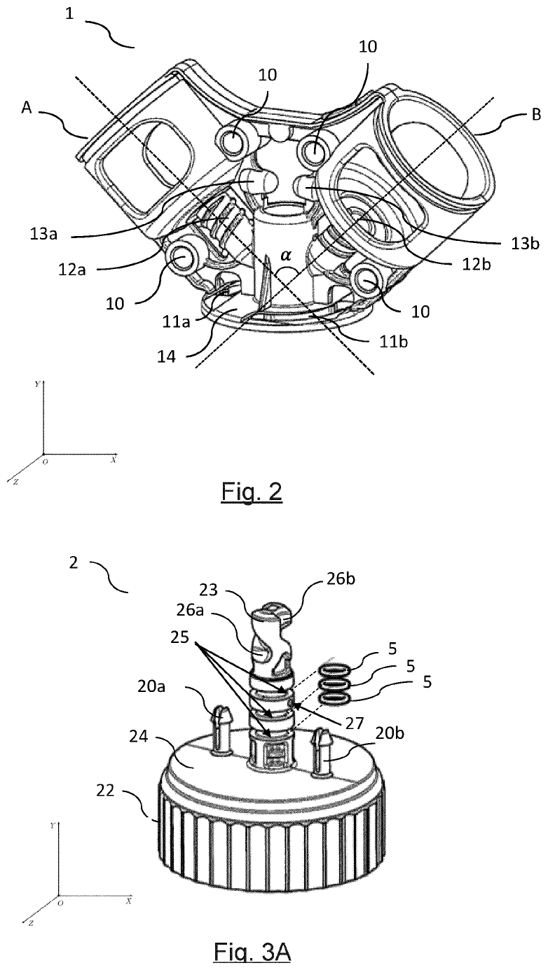

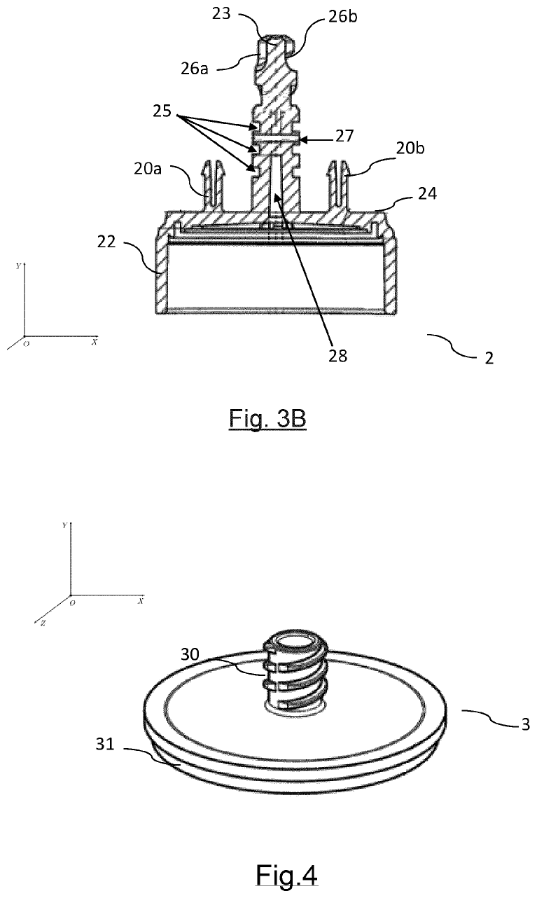

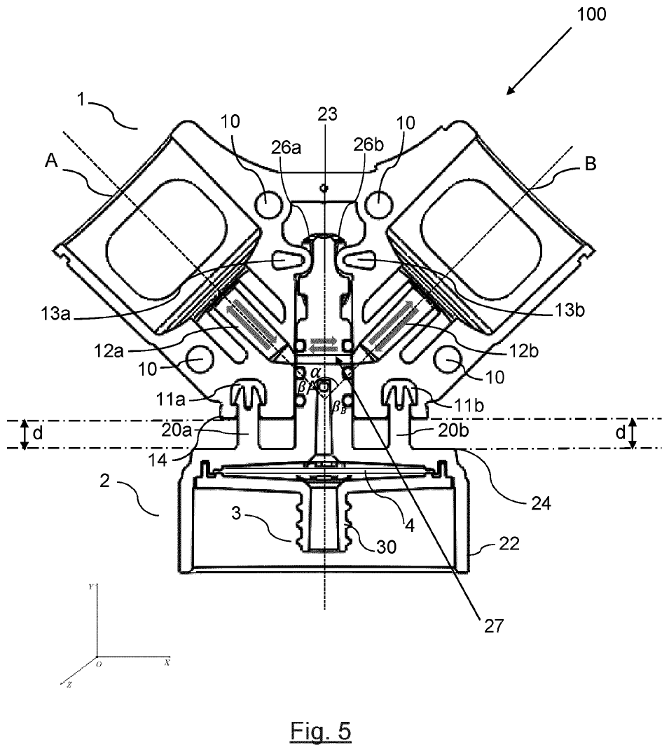

Among these elements, there are at least three different main elements: a frame 1, a pin 2 and a cover 3, which are described below, with reference to FIG. 2, FIG. 3A, FIG. 3B, and FIG. 4 respectively.

In particular, the pin 2 fits into the frame 1 and the cover 3 fits into the pin 2. More particularly, the pin 2 is secured to the frame 1 in such a way that at least part of the pin 2 passes through the frame 1 along its longitudinal axis. Thus, the longitudinal axes of the pin 2 and the frame 1 coincide. In other words, if we consider a standard orthonormal frame of reference in the three directions xyz, the pin 2 is fitted into the frame 1 along the same vertical axis y.

The cover 3 is secured to the pin 2 such that its longitudinal axis y coincides with the longitudinal axis y of the pin 2.

The pin 2 is mounted underneath the frame 1 and the cover 3 is mounted underneath the pin 2 along a vertical axis y. Furthermore, the pin 2 is movable relative to the frame 1 and configured to be actuated by a practitioner. More specifically, from the effect of such an action (such as for example the application of a rotational movement about the y axis of the pin 2 by the practitioner in clockwise or anticlockwise direction), the pin 2 can assume two different operating positions relative to the frame 1. These two positions of the pin 2 correspond to the first emulsification position and the second transfer position of the emulsified fat.

The preparation device 100 further comprises a shell 6 formed by a first part 6a and a second part 6b. These two parts 6a and 6b fit into one another by means of a plurality of studs 60a and 60b (not shown) located on the inner face of the first and second parts 6a and 6b of the shell 6. The studs on the inner phase of the first part 6a are complementary to the studs on the inner face of the second part 6b and enable the shell 6 to be fixed around the frame 1. The studs 60a and 60b of the shell 6 pass through openings 10 made in the frame 1. Once assembled in the frame 1, the first and second parts 6a and 6b of the shell 6 surround the latter to protect it from possible damage, for example during the transport of the preparation device 100 or even mishandling by a practitioner (for example dropping, impact etc. . . . ).

The preparation device 100 also comprises a filtering membrane 4 positioned in the pin 2 and held in place by the cover 3. This filtering membrane 4 is used to filter the fat after emulsification when it is transferred for recovery of the nano-fat, prior to autologous grafting on a patient.

The filtering membrane 4 is for example a flexible PET (polyethylene terephthalate) filtering membrane. Filtration through this type of flexible membrane is less stressful or traumatising for the elements making up the nano-fat which contributes to a better quality nano-fat at the end of the emulsification/filtration process using the preparation device 100.

In one embodiment of the invention, the filtering membrane 4 has a mesh size of between 420 μm and 460 μm, preferably 440 μm. It is thus possible to obtain nano-fat without having to successively change filter sizes in order to progressively reduce the size of the fat. In other words, a single pass of the emulsified fat through the filtering membrane 4 is necessary to obtain nano-fat.

FIG. 2 shows a diagram of the frame according to one embodiment of the invention.

The frame 1 is a structure to which two standard syringes can be attached, in particular syringes with a Luer tip, the pin 2 and the shell 6. In other words, the syringes, the pin 2 and the shell 6 are mounted around this central structure 1 of the preparation device 100.

In one embodiment of the invention, the frame 1 is a rigid structure in the form of a monobloc, i.e. a single piece. The frame 1 is made of rigid plastic for example.

In particular, the frame 1 allows two syringes to be connected to a first branch A and a second branch B which are fixed to the frame in a conventional manner, for example via a Luer tip system. The first branch A and second branch B are symmetrical to one another along the longitudinal axis y of the frame 1.

In other words, the two syringes are connected to one another via the pin 2 by way of the frame 1.

To achieve this, the first branch A and the second branch B of the frame 1 each comprise a syringe connection port 12a and 12b in the form of a female Luer lock onto which the male Luer lock of the syringes is attached by rotation. The Luer lock is a standardised system of connectors for small fluids used to form non-leaking connections between a male cone connector and its corresponding female part.

In one embodiment of the invention, the first and second branches A and B of the frame 1 each further comprise at least one protuberance, also denoted as followers 13a and 13b, which each engage in a complementary manner in movement interfaces of a central stud of the pin 2.

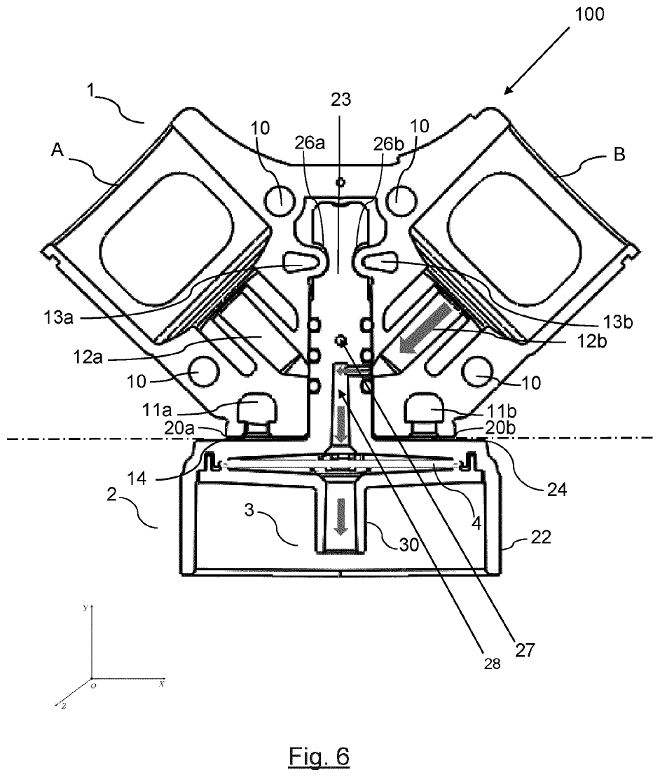

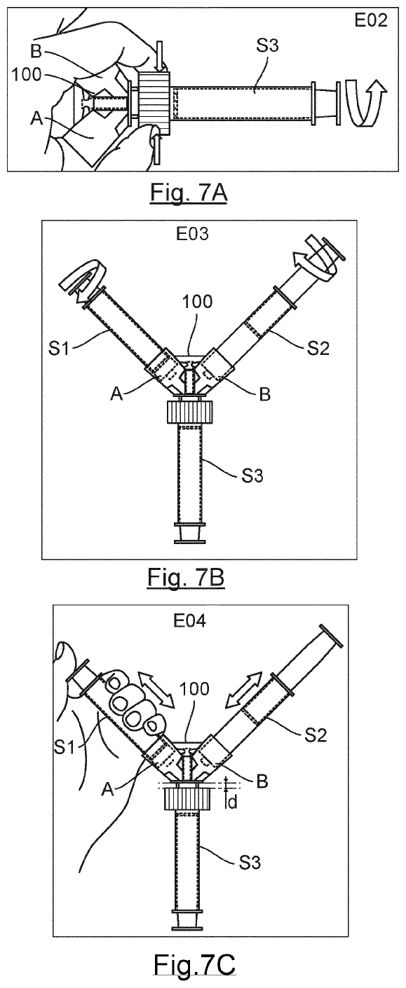

In one embodiment of the invention, these two followers 13a and 13b, once engaged in the movement interfaces of the pin 2, make it possible in collaboration with the movement interfaces of the pin 2, to convert a rotational movement of the pin 2 about its longitudinal axis y into a translational movement according to the y axis in a standard orthonormal frame of reference. In other words, if we consider that the frame 1 is fixed relative to the practitioner, then the followers 13a and 13b, once engaged in the movement interfaces of the pin 2, enable a clockwise or anticlockwise rotational movement about the y axis of the pin 2 relative to the frame 1 to be converted into an up/down movement along the y axis of the pin 2 relative to the first and second branches A and B of the frame 1. As a result, the pin 2 can assume two different positions relative to the frame 1:
a lower or remote position of the frame 1 corresponding to the first emulsification position of the fat, in which the first and second branches A and B, i.e. the two syringes are connected, and
a high or close position of the frame 1 corresponding to the second fat transfer position, in which the first and the second branches are disconnected, and at least one of these branches is connected to another syringe connected to the pin 2. Details of the arrangement of the frame 1 and the pin 2 in relation to the different positions are described with reference to FIG. 5 and FIG. 6.

In one variant, the practitioner applies a translational movement relative to the frame 1 to the pin 2 along the vertical axis y and it is the followers 13a and 13b of the branches A and B, which, once engaged in the movement interfaces of the pin 2, make it possible to hold the latter in one of the first or second emulsification or transfer positions. In other words, the followers 13a and 13b slide in the corresponding movement interfaces of the pin 2 along a vertical axis y, the positions being maintained for example by means of notches or slots in the movement interfaces of the pin 2.

Furthermore, the frame 1 comprises a base 14 on which the first and second branches A and B are mounted. These first and second branches A and B are then positioned relative to one another on this base 14 in the form of a V. In other words, the angle α between the longitudinal axis passing through the centre of the first branch A and the longitudinal axis passing through the centre of the second branch B is between 45° to 135°, preferably 90°. Thus, during a standard emulsification step with successive back and forth movements in two syringes, the practitioner's wrists positioned substantially between 45° to 135°, preferably 90°, relative to one another. The practitioner's hands are therefore positioned in a much more comfortable and ergonomic way, which reduces fatigue and the risk of injury.

The base 14 of the frame 1 comprises at least one, preferably two tracks 11a and 11b. These first and second tracks 11a and 11b are for example in the form of openings in the form of an arc of a circle, positioned opposite each other on the outer part of the base 14. For example, the tracks 11a and 11b have a length corresponding to a quarter of a circle and are positioned opposite one another symmetrically relative to the centre of the base 14 of the frame 1.

In this embodiment of the invention, the tracks 11a and 11b comprise a constriction at both ends enabling the pin 2 to be held in the emulsification position or in the transfer position. The change from one position to another is reversible and is achieved by simply disengaging the pin 2 from either of the ends of the tracks 11a and 11b. In other words, the tracks 11a and 11b each comprise a first end for engaging the emulsification position and an end for engaging the transfer position. The change from one position to the other is performed by removing the pin 2 from the constriction at one end of one of the tracks 11a or 11b to fit into the constriction of the other end of the tracks 11a or 11b.

These first and second tracks 11a and 11b thus allow the pin 2 to be connected to the frame 1 while allowing a rotational and/or translational movement in the y axis of the pin 2 relative to the frame 1.

In one variant, the track or tracks 11a and 11b are simple circular openings, i.e. holes traversing the base 14 of the frame 1.

A diagram and longitudinal cross-section of the pin according to one embodiment of the invention are now described respectively with reference to FIG. 3A and FIG. 3B.

The pin 2 is element for connecting various parts of the preparation device 100. In other words, the pin 2 is used to either connect the two syringes fixed to the first and second branches A and B of the frame 1 to one another, or to connect one of these syringes to another syringe fixed to the skirt 22 of the pin 2 via the cover 3. In particular, it is the change in position (from the emulsification position to the transfer position and vice-versa) of the pin 2 which enables two different circuits to be created for circulating the flow of fat and therefore connect the different syringes two-by-two according to these different circuits.

In one embodiment of the invention, the pin 2 comprises a base 24 supporting at least one, preferably two fixing studs 20a and 20b, or annular clips, fixed to the base 24 at a first end. The fixing studs 20a and 20b are, for example, substantially cylindrical with a cone-shaped flare at a second end. The fixing studs 20a and 20b fit respectively into the tracks 11a and 11b of the frame 1. The conical flare of the fixing studs 20a and 20b makes it possible to secure the pin 2 to the frame 1. More particularly, the particular form of the fixing studs 20a and 20b first of all makes it possible for the pin 2 to be fitted into the tracks 11a and 11b of the frame 1 and then prevents the pin 2 from coming loose. Thus, the fixing studs 20a and 20b allow the pin 2 to be assembled and secured to the frame 1.

The base 24 of the pin 2 comprises a grooved skirt 22 to make it easier for the pin 2 to be gripped by the practitioner.

Once positioned in the tracks 11a and 11b, the fixing studs 20a and 20b also allow the pin 2 to move relative to the frame 1 (if you consider that the frame 1 is fixed relative to the practitioner).

The pin 2 comprises a central stud 23 on its base 24. In one embodiment, the central stud 23 is located between the two fixing studs 20a and 20b. The central stud 23 is larger in size than the fixing studs 20a and 20b and passes through the frame 1 between the two branches A and B. The central stud 23 comprises at least one, preferably three circular grooves 25 into which seals 5 are placed. These seals 5 ensure that the preparation device 100 is sealed during the emulsification and transfer steps.

The pin 2 comprises at least one, preferably two movement interfaces 26a and 26b. In one embodiment of the invention, these two movement interfaces 26a and 26b allow the rotation of the pin 2 relative to the frame 1 to be converted into a translational movement of the latter in the frame 1 along the longitudinal axis of the pin 2 and the frame 1. For example, these movement interfaces 26a and 26b, or movement conversion interfaces, are in the form of substantially semi-helical grooves located at the end of the central stud 23 furthest from the base 24, 25 and are located opposite one another. It is this particular form of the grooves which enables the rotational movement to be converted into a translational movement. The followers 13a and 13b of the branches A and B engage in these two opposite grooves. Thus, when the practitioner applies a rotational movement to the pin 2, for example by turning the pin 2 a quarter turn relative to the frame 1 (rotation about the longitudinal axis y), the two followers 13a and 13b follow the semi-helical grooves 26a and 26b, which allows the central stud 23, and therefore by extension the entire pin 2, to move in translation along the y axis, which has the effect of moving the pin 2 away from or towards the frame 1. The fixing studs 20a and 20b then slide in the tracks 11a and 11b of the frame 1 in a reversible manner to a locking position located at one or other of the ends of the tracks 11a and 11b. In one variant, the grooves, or movement interfaces 26a and 26b, are rectilinear along a vertical axis y of the pin 2 and comprise notches or slots. The followers 13a and 13b then engage in the notches of the grooves 26a and 26b to hold the pin 2 in one position or the other. The position of the pin 2 is changed by simple translation along the y axis of the pin 2 relative to the frame 1. The central stud 23 also comprises a first channel for the passage of fat 27 which passes through it in a transverse manner, i.e. along an x axis. The central stud 23 also comprises a second fat transfer channel 28 which traverses the central stud 23 in a transverse manner only on one part of the latter.

In one embodiment the first channel 27 is positioned above the second channel 28 in the central stud 23, the inlet of the second channel 28 being offset by a quarter turn about the vertical axis y relative to the traversing openings in the first channel 27.

In one variant, the first channel 27 is positioned above the second channel 28 in the central stud 23, the inlet of the second channel 28 being aligned with one of the traversing openings of the first channel 27.

FIG. 3B is a longitudinal cross-section of the pin 2. The first fat transfer channel 27 of the central stud 23 makes it possible to connect the two syringes fixed to the first and second branches A and B of the frame 1 during the emulsification step. The second fat transfer channel 28 of the central stud 23 is hollowed out inside the central stud 23, firstly transversely (along the horizontal x axis), then longitudinally (along the vertical axis y). Advantageously, the second channel 28 is substantially in the form of an inverted L hollowed out inside the central stud 23. In other words, the channel 28 forms a bend inside the stud 23. This second channel 28 makes it possible to connect one of the syringes fixed to the first branch A or the second branch B of the frame 1 to a third syringe connected to the lower end of the pin 2 during the transfer step.

In one embodiment of the invention, the channel 28 connects in particular branch B to the pin 2, it is thus not possible to make a transfer between the syringe connected to branch A to the third syringe connected to the pin 2.

Thus, when the pin 2 is in the emulsification position, the connecting ports 12a and 12b of the frame 1 are opposite the first passage or emulsification channel for the fat 27 to form a first circulation circuit for the flow of fat between two syringes fixed to the connecting ports 12a and 12b via the passage channel 27.

Conversely, when the pin 2 is in the fat transfer position, only one of the connecting ports 12a or 12b is opposite the second fat transfer channel 28 to form a second circulation circuit for the flow of fat between one of the syringes fixed to one of the connecting ports 12a or 12b and a third 25 syringe connected to the lower end of the pin 2 via this second channel 28.

A diagram of a cover according to one embodiment of the invention is now described with reference to FIG. 4.

The cover 3 comprises a connecting port, or female Luer tip 30 allowing a syringe with a male Luer tip to be attached to the cover 3.

The cover 3 comprises a skirt 31 making it possible for it to fit into the pin 2 on the face of the base 24 of the pin 2 opposite the one comprising the fixing studs 20a and 20b and the central stud 23, or said lower face of the pin 2.

In one embodiment of the invention, the cover 3 is fixed irreversibly to the pin 2, for example by ultrasonic welding. The filtering membrane 4 is then fitted into the pin 2 by fixing the cover 3. In other words, the filtering membrane 4 is sandwiched between the pin 2 and the cover 3.

In one variant, the cover 3 is fixed in a reversible manner, for example by means of a complementary thread between the pin 2 on the face of the base 24 of the pin 2 opposite the one comprising the fixing studs 20a and 20b and the central stud 23 and the skirt 31 of the cover 3 and a seal. Advantageously, it is possible to change the filtering membrane 4 and reuse the preparation device 100.

Thus, the pin 2 forms a third branch of the fat preparation device 100 which comprises a filtering membrane held by the cover 3, the first and second branches being the branches A and B of the frame 1.

As described above, the preparation device 100 makes it possible to recover the nano-fat from the fat harvested by liposuction for example in a lipostructure procedure. In particular, in order to recover the nano-fat, the preparation device 100 according to the invention makes it possible to emulsify the fat and filter it inside a single fat preparation device.

To achieve this, the preparation device 100 is configured to assume two positions:
the first position, referred to as the emulsification position, for the emulsification step by successive back and forth movements of the fat in two standard syringes and in which the connecting element 2 (i.e. the pin 2) forms with the first and second branches A and B of the frame 1 a first circulation circuit of the fat between a first syringe and a second syringe fixed to the frame 1 via the branches A and B;
the second position, referred to as the transfer position, for transferring fat from one syringe to another through a filtering membrane 4 and in which the pin 2 forms with one of the first or second branches A and B of the frame 1 a second circuit for circulating fat between the first or the second syringe and a third syringe fixed via the cover 3 to the lower face of the pin 2.

It is the movement of the pin 2 relative to the frame 1 which allows this change in position. In particular, as described above, the pin 2, due to the fixing studs 20a and 20b which slide in the tracks 11a and 11b of the frame 1, can make a quarter turn clockwise or anti-clockwise relative to the base 14 of the frame 1 about the y axis. The followers 13a and 13b then follow the semi-helical movement conversion interfaces 26a and 26b of the central stud 23 of the pin 2 which transforms the rotational movement into a translational movement of the pin 2 along the y axis. Consequently, the pin 2 moves from the first emulsification position to the second transfer position (and vice versa) by rotation about the y axis and simultaneous translation along this axis.

One advantage of such a preparation device 100 based on the conversion of f a rotational movement to a translational movement is that it is simple to use, i.e. the practitioner only has to make a limited number of movements to carry out the first emulsification operation, change the position of the device and then carry out the second filtration/transfer operation, but is also simple to make. To provide such a preparation device, it is not necessary to assemble a plurality of complex parts. On the contrary, a limited number of simple parts is sufficient.

Furthermore, the preparation device 100 according to the invention is particularly ergonomic and also guarantees that a nano-fat of excellent quality is obtained.

A diagram of the fat preparation device in the emulsification position according to one embodiment of the invention is now described with reference to FIG. 5.

During the emulsification step, the connecting element 2, i.e. the pin, is in the emulsification position relative to the frame 1. More specifically, in the emulsification position, the base 24 of the pin 2 is at a distance d, for example 4 to 5 mm, preferably 4.4 mm, from the frame 1, i.e. there is gap, or clearance, between the base 24 of the pin 2 and the base of the frame 1. Due to the particular shape of the fixing studs 20a and 20b which slide in tracks 11a and 11b in the base 14 of the frame 1, the pin 2 is mobile relative to the frame 1, while remaining integral therewith.

In the emulsification position, the syringe connecting ports 12a and 12b can communicate with each other via the channel for the passage of fats 27 located in the central stud 45 of the pin 2. Thus, the flow of fat (represented by the grey arrows) to be emulsified can pass from a first syringe connected to a first connecting port 12a to a second syringe connected to a second connecting port 12b. The successive passage of the fat from one syringe to the other enables the fat to be emulsified in order to obtain nano-fat, for example after 30 back and forth movements by the practitioner applying pressure to one or other of the plungers of the syringes.

Advantageously, the first branch A of the frame 1 forms an angle β_{A} with the pin 2 of approximately 112.5°-157.5°, preferably 135°, and the second branch B of the frame 1 forms an angle β_{B}with the pin 2 of approximately 112.5°-157.5°, preferably 135°. Thus, the preparation device 100 is more ergonomic to use. The angle β_{A} is the angle between the longitudinal axis passing through the centre of the first branch A and the longitudinal axis passing through the centre of the pin 2, and the angle β_{B} is the angle between the longitudinal axis passing through the centre of the first branch B and the longitudinal axis passing through the centre of the pin 2.

In other words, the preparation device 100 according to the invention comprises three branches: a first branch A of the frame, a second branch B of the frame and a third branch formed by the pin 2. These three branches are then positioned relative to one another in the form of a Y to ensure the best possible ergonomics.

A diagram of the fat preparation device in transfer position according to one embodiment of the invention is now described with reference to FIG. 6.

During a fat transfer step, the connecting element 2, or pin, is in a so-called fat transfer position relative to the frame 1. More particularly, in the transfer position, the base 24 of the pin 2 is no longer at a distance d (d=0) from the base 14 of the frame 1, i.e. there is no longer any space between the base 24 of the pin 2 and the base 14 of the frame 1.

Furthermore, one of the branches A or B of the frame 1 is then connected to the fat transfer channel 28. Thus, emulsified present at the end of the the fat emulsification step in one of the syringes, known as the transfer syringe, passes through the connecting port of the latter then the transfer channel 28 to end up in the syringe fixed to the cover 3 or recovery syringe.

In this example, it is the connecting port 12b which is then connected to the fat transfer channel 28. Thus, the emulsified fat can be transferred from the transfer syringe connected to the connecting port 12b to a third syringe connected to the connecting port 30 of the cover 3 via the fat transfer channel 28. So that the practitioner can identify which branch of the frame is the transfer branch, i.e. which one the transfer syringe is fixed to, an arrow is shown on the transfer branch.

The emulsified fat then passes through the filtering membrane 4 sandwiched between the pin 2 and the cover 3.

In one variant, the practitioner manipulates the pin 2 by applying a longitudinal translational movement thereto (along the y axis) relative to the frame 1 to pass from one position to the other. In other words, the practitioner moves the pin 2 away from or towards the frame in the direction of the longitudinal axis y, raising or lowering it relative to the frame 1.

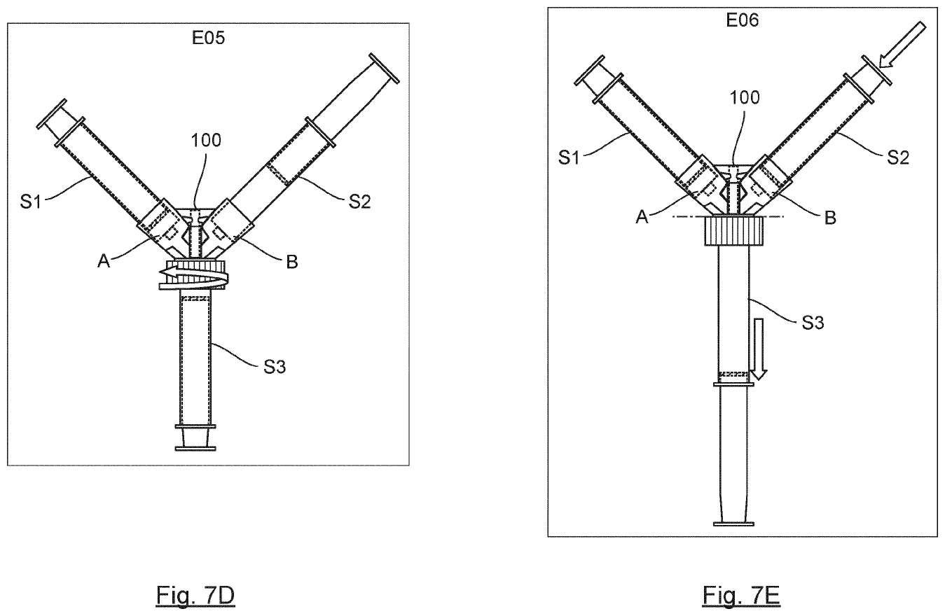

Diagrams of different steps of the emulsification/filtration process using the fat preparation device according to one embodiment of the invention are now described with reference to FIGS. 7A to 7E.

In step E01 (not shown), during a lipostructuring procedure, the practitioner recovers the patient's fat in the usual way. This fat may also be prefiltered according to a known filtration technique to remove all the particles which are not fat or fatty cells and to recover micro-fat in particular.

In step E02, with reference to FIG. 7A, the practitioner attaches a standard syringe S3 to the connecting port 30 of the cover 3 of the preparation device 100. To achieve this, the practitioner can hold the preparation device 100 by the skirt 22 of the pin 2. Then the male Luer tip of the syringe S3 is screwed onto the female Luer tip 5, or connecting port 30 of the cover 3 associated with the pin 2 on the underside of the pin 2.

In step E03, with reference to FIG. 7B, the practitioner then screws the male Luer tip of the two other syringes S1 and S2 onto the connecting ports 12a and 12b of branches A and B of the preparation device 100. One of these syringes, for example syringe S2 contains the previously harvested and possibly prefiltered, which needs to be emulsified to recover the nano-fat.

In step E04, as shown in FIG. 7C, the practitioner positions the pin 2 in the emulsification position. More particularly, the practitioner turns, for example by a quarter turn, the pin 2 until the fixing studs 20a and 20b of the pin 2 slide into the tracks 11a and 11b of the frame 1 up to the constriction located at one of the ends of the tracks 11a and 11b to clip the fixing studs 20a and 20b into the tracks 11a and 11b and thus maintain the emulsification position. The rotational movement about the y axis of the pin 2 is then transformed simultaneously into a translational movement along this axis of the pin assembly 2 by sliding the followers 13a and 13b into the movement conversion interfaces 26a and 26b of the central stud 23. The base 24 of the pin 2 is then separated from the base of the frame 1 by a distance d. In other words, the pin 2 turns and descends relative to the frame 1 along the y axis. The central stud 23 therefore turns a quarter turn and descends along the y axis inside the frame 1.

In this emulsification position, the fat passage channel 27 or emulsification channel 25 of the central stud 23 of the pin 2 is then opposite the connecting ports 12a and 12b of the frame 1, thus forming a first fat circulation circuit.

The practitioner can then proceed to emulsify the fat by successive back and forth movements of the fat in the syringes S1 and S2 in a conventional manner.

In step E05, with reference to FIG. 7D, the practitioner changes the position of the pin 2 of the preparation device 100 to a fat transfer position. To achieve this, the practitioner turns the pin 2, for example by a quarter turn, for example in opposite direction than before, until the fixing studs 20a and 20b of the pin 2 slide into the tracks 11a and 11b of the frame 1 up to the constriction at the ends of the tracks 11a and 11b which are opposite the ends for maintaining the emulsification position and to clip the fixing studs 20a and 20b into the tracks 11a and 11b in order to maintain the transfer position during the transfer step. The rotational movement of the pin 2 about the y axis is then transformed simultaneously into a translational movement along this axis of the pin 2 by sliding the followers 13a and 13b into the movement transformation interfaces 26a and 26b of the central stud 23. In other words, the pin 2 turns and moves up in relation to the frame 1 according to the y axis. The central stud 23 therefore turns a quarter turn in opposite direction than before about the y axis and moves up along this axis thus rising inside the frame 1.

In this transfer position, the base 24 of the pin 2, is no longer separated by a distance d (d=0) from the base 14 of the frame 1, but in contact with it. The emulsified fat contained in one of the syringes fixed to the branches of the frame 1 can then be transferred into the syringe fixed to the cover 3. More particularly, in this transfer position the transfer channel 28 of the central stud 23 of the pin 2 then faces one of the connecting port 12a or 12b of the frame 1 thus forming a second circulation circuit for the emulsified fat.

In other words, one of the syringes S1 or S2 is a so-called emulsified fat transfer syringe, i.e. at the end of the emulsification step, the practitioner ensures that all the emulsified fat is in a single syringe (identified by an arrow on the transfer branch) which can be connected to the emulsified fat recovery syringe connected to the cover 3 via the transfer channel 28.

In step E06, with reference to FIG. 7E, the practitioner then applies pressure to the plunger of the transfer syringe, for example the syringe S2 connected to the branch B of the frame 1. The flow of emulsified fat therefore passes from the syringe S2 to the connecting port 12b, then through the transfer channel 28 of the pin 2 to end its journey in the recovery syringe S3 connected to the cover 3. During the transfer, the emulsified fat 15 passes through the filtering membrane 4 located in the pin 2 and held by the cover 3.

The nano-fat obtained by emulsification, then filtration by the preparation device 100 is then recovered in the syringe S3 and is ready for reinjection into the patient in a conventional manner.

The particular form of the preparation device 100 according to the invention (in a Y shape, once all the elements have been assembled) allows the practitioner's hands and wrists to be in a better position and therefore facilitate the emulsification step by successive back and forth movements of the fat. The presence of a filtering membrane in one of the branches of the device (formed by the pin 2) makes it possible to filter the fat directly after emulsification, without having to change the device and therefore remaining in a closed circuit. Thus, the preparation device 100 makes it possible to reduce the risk of contaminating the nano-fat obtained. Furthermore, integrating the filter directly into a third branch (formed by the pin 2) of the preparation device 100 makes it possible to limit the risk of leaks, associated in particular with a poor filter connection.

1. A fat preparation device for preparation of fat harvested from a first area of a patient's body for reinjection into a second area of the patient's body, said fat preparation device comprising:
a structure comprising a first and a second branch to which a first syringe and a second syringe are connected respectively; and
a connecting element of said first and second branches, said connecting element fitting into said structure and comprising a membrane for filtering said fat, and said connecting element being connected to a third syringe and configured to assume at least two positions:
an emulsification position in which said connecting element forms with said first and second branches of said structure a first circulation circuit for the fat between the first syringe and the second syringe, and
a transfer position in which said connecting element forms with said first or second branch of said structure a second circulation circuit for the fat between the first or the second syringe and a third syringe.

2. The fat preparation device according to claim 1, wherein said first and second branches of said structure are positioned relative to one another in the form of a V.

3. The fat preparation device according to claim 2, wherein said first and second branches of said structure are positioned relative to one another at an angle between 45° and 135°.

4. The fat preparation device according to claim 2, wherein said first and second branches of said structure form a Y shape with said connecting element.

5. The fat preparation device according to claim 4, wherein said first branch forms a first angle with said connecting element of approximately 112.5°-157.5°, and said second branch forms a second angle with said connecting element of approximately 112.5°-157.5°.

6. The fat preparation device according to claim 1, wherein said filtering membrane is held in said connecting element by a cover fixed to said connecting element, said cover comprising a connecting port with said third syringe.

7. The fat preparation device according to claim 1, wherein said structure comprises at least one track comprising a first end for engaging the emulsification position and a second end for engaging the transfer position and wherein said connecting element comprises a base with at least one fixing stud, said at least one fixing stud being adapted to fit into and slide in said at least one track until said at least one fixing stud locks into either of the first and second ends, when the connecting element is placed in the corresponding position.

8. The fat preparation device according to claim 7, wherein said connecting element comprises a central stud fixed to said base and oriented in a longitudinal axis of said structure, said central stud comprising at least two movement transformation interfaces into which at least one follower of said first branch and at least one follower of said second branch of said structure slide to transform a rotational movement of said connecting element relative to said structure, by an effect of a rotational action of said connecting element exerted by a practitioner on said base about said longitudinal axis, into a translational movement of said connecting element in said longitudinal axis.

9. The fat preparation device according to claim 8, wherein said central stud further comprises:
a first fat emulsification channel which forms with said first and second branches of said structure said first circulation circuit of said fat between said first syringe and said second syringe, when said connecting element is placed in said emulsification position,
a second fat transfer channel which forms with said first or second branch of said structure said second circulation circuit of fat between the first or second syringe and said third syringe, when said connecting element is placed in said transfer position.

10. A method for preparation of fat harvested from a first area of a patient's body for reinjection into a second area of said patient's body, wherein the method comprises:
using a fat preparation device to prepare the fat, the device comprising:
a structure comprising a first and a second branch to which a first syringe and a second syringe are connected respectively; and
a connecting element of said first and second branches, said connecting element fitting into said structure and comprising a membrane for filtering said fat, and said connecting element being connected to a third syringe and configured to assume at least two positions:
an emulsification position in which said connecting element forms with said first and second branches of said structure a first circulation circuit for the fat between the first syringe and the second syringe, and
a transfer position in which said connecting element forms with said first or second branch of said structure a second circulation circuit for the fat between the first or the second syringe and a third syringe;
positioning the connecting element in said emulsification position;
emulsifying said harvested fat by successive and repeated pressing movements by an operator on said first and second syringes, said pressing movements causing said fat to circulate in said first circuit from said first syringe to said second syringe and vice-versa;
positioning said connecting element in said transfer position by changing the position of said connecting element relative to said structure;
filtering and transferring said fat emulsified by the operator pressing on one of the first or second syringes, said pressing movement causing said harvested fat to flow in said second circuit through said filtering membrane into said third syringe.

11. The fat preparation device according to claim 2, wherein said first and second branches of said structure are positioned relative to one another at an angle of 90°.

12. The fat preparation device according to claim 4, wherein the first angle and the second angle are 135°.
